(19) United States
(12) Patent Application Publication
(10) Pub. No.: US 2011/0092928 A1
(43) Pub. Date: Apr. 21, 2011

(54) ASSEMBLY FOR COLLECTING URINE FROM A MALE

(76) Inventor: Jose M. Saez, Milford, DE (US)

(21) Appl. No.: 12/581,354

(22) Filed: Oct. 19, 2009

(51) Int. Cl. A61M 1/00

(52) U.S. Cl. 604/326

(57) ABSTRACT

An assembly for collecting urine from a male patient includes a generally rigid shape retaining support member which would be disposed against the shaft of the penis of a male patient. Attaching structure such as straps mount the support member to the shaft. A condom catheter is then placed over the support member and over the penis for collecting urine from the penis.

### BACKGROUND OF INVENTION

There is currently concern for the need to curb healthcare costs. Various medical specialists care for patients on a daily basis and attempt to discharge the patients in a timely fashion. There is also a strong concern to limit infections in the hospital. For example, certain insurance guidelines will not authorize payment for patients that are admitted to a hospital and acquire an infection while hospitalized. Examples of such infections could be hospital acquired are pneumonia and urinary tract infections.

In certain medical practices, such as performed by a kidney specialist, one of the daily pieces of information required is to document accurate urinary output in patients who present or developed renal failure during the hospitalization. This has been done by placing an indwelling foley catheter into the urethra up into the urinary bladder. Patients who are hospitalized are usually immunocompromised due to their illness. Such patients are at increased risk of developing urinary tract infections because of the indwelling foley catheter which is a foreign object to the patient.

Another type of catheter that has been used to collect urine without having a foreign object in the urinary bladder is referred to as a Texas catheter or condom catheter. A problem with a condom catheter, however, is that it is not effective because it does not tend to stay on the patient's penis which would not normally be erect.

### SUMMARY OF THE INVENTION

An object of this invention is to provide an assembly for collecting urine from a male patient which overcomes the above disadvantages.

In accordance with this invention the assembly includes a generally rigid shape retaining support member which would be disposed against the penis of a male patient. The support member is attached to the shaft of the penis. A condom catheter is then placed over the support member and the penis for collecting the urine from the penis, or preferably a human male patient.

In a preferred practice of the invention the support member is generally concave and includes two sets of slits. The attaching structure for mounting the support member to the shaft of the penis is in the form of a pair of soft flexible straps which preferably have hook/loop formations. Each strap is preferably anchored at one end to the support member and then inserted through one slit and placed around the penis. Finally, the strap is inserted through the opposite slit of its set of slits and engaged with the anchored end to be held in place. The condom catheter is then placed over the penis which is in effect in an erect stiff condition because of the attached support member.

### THE DRAWINGS

FIG. 1 is a side elevational view showing an assembly for collecting urine from a male in accordance with this invention; and

FIG. 2 is a plan view of the support member shown in FIG. 1.

### DETAILED DESCRIPTION

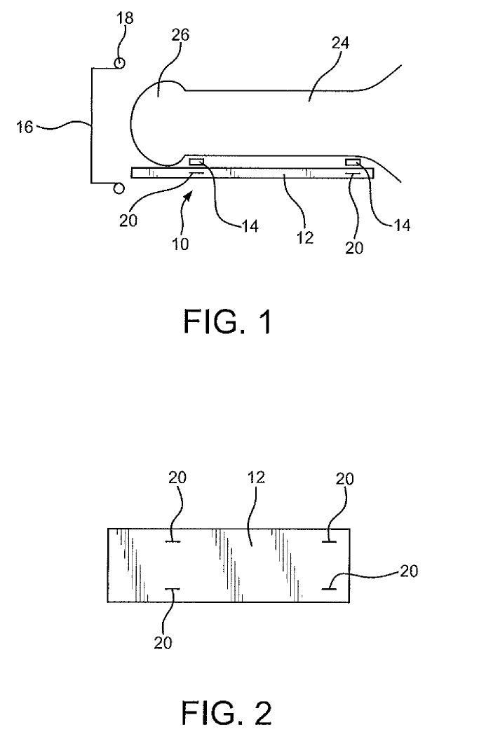

The present invention is based upon the realization that condoms are not meant to be placed and, more importantly, to stay on a non-erect penis. In accordance with this invention an assembly is provided which in effect will erect the penis so that a condom catheter will stay on without difficulty. As shown in FIG. 1 the assembly 10 includes as its components a support member 12 and attaching structure in the form of straps 14,14. The assembly also includes a condom catheter 16 which could be of known conventional construction. In that regard, the condom catheter 16 could be an elastic flexible sheet initially rolled at its end 18 so that it can then be unrolled when it is mounted in place.

As shown in FIG. 2 support member 12 is generally of rectangular shape which, in the preferred practice of this invention, is 4.5 inches long and 1 inch wide. Other lengths and widths could be used within the practice of this invention. For example, the length could be within the range of 3.5-5.5 inches and the width could be in the range of ¾-1¼ inches. Support 12 includes two sets of slits 20 cut through the support.

Preferably, support 12 is concave in shape to generally conform to the shaft 24 of the penis located below the glans penis 26. The concave shape permits the support 12 to generally conform to the penis and is of a length which would extend along the shaft 24 to generally being along the glans penis 26. Support member 12 is preferably made of a rigid material which his shape retaining such as a suitable plastic. The support member 12 could be slightly bendable without departing from the broad practice of this invention.

The attaching structure 14 is preferably a pair of flexible straps 14 which is of appropriate thickness to be able to be inserted through the slits 20 and then wrapped around the shaft 24 and yet be of sufficient strength to effectively mount the support 12 to the shaft 24. Straps 14 should be of sufficient length so that one end of each strap could be anchored to one side of the support member 12. The strap will then be inserted through one of a set of slits 20 and would wrap around the shaft 24 and then be inserted through the opposite slit in its set of slits 20 to be anchored in place. Preferably, each strap includes fastening structure such as Velcro which utilizes hook/loop formations. Where such hook/loop formations are used the anchored end of the strap is preferably anchored to the outer surface of support member 12 at a location between the two slits 20 of its set of slits so that the opposite free end of the strap 14 could then be disposed against the anchored end and securely held in place by the engagement of the hooks and loops. Preferably the hook/loop formations are on the outer surface of the anchored end and on the inner surface of the free end of each strap. Straps 14 could be of any appropriate suitable dimensions such as being ¾ inches in width.

After the support member 12 is mounted in place through use of the attaching structure for straps 14, the condom catheter 16 is rolled over the glans penis and then simultaneously rolled over the support member 12 and shaft 24. By providing one strap at the tip of the penis near the glans penis and providing the other strap at the base of the shaft, the penis is held stretched out or erect. As a result the condom catheter 16 will stay on the penis and collect urine without the risk of infection.

The above description sets forth the preferred manner of practicing the present invention. It is to be understood that variations could be used without departing from the spirit of the invention. For example, in the preferred practice of this invention only a single support member is used. Two or more support members could also be used to provide stiffness at different locations around the shaft. A single support member, however, is preferred since it would provide a greater area of contact of the straps 14 with the shaft 24.

Any suitable materials could be used for making the components of assembly 10. Support member 12 is preferably made of a plastic having its edges rounded. In particular the ends of the support member 12 should be rounded or dull so as not to be sharp.

Although the use of hook/loop formations is the preferred manner of attaching the straps to the support, other types of structures can be used wherein a free end of the strap would be engaged with either the support member or a different portion of the same strap, such as an arrowhead shaped free end inserted into a slit. Attaching structure could be provided on the support member 12, itself, for engagement by the free end of the strap. Preferably, the anchored end of the strap is anchored to the outer side of the support member 12. The invention, however, could be practiced where one end of the strap 14 is anchored to the inner side and then the strap wraps around the shaft 24 and is inserted through a slit 20 in the support member 12 and anchored to permanent anchoring structure on the outer side of the support member 12. In such practice of the invention there need only be one slit in each set at each end of the support member 12.

As shown in FIG. 1, the support member 12 has a tip end generally near the glans penis 26 and has a base end at the base of the shaft 24. In the preferred practice of this invention two straps 14 are used. One strap is at the tip end of the support member and the other strap is at the base end. If desired one or more intermediate straps and sets of slits may also be used.

In the preferred practice of this invention one end of strap 14 is anchored to support member 12 at a location between the first and second slits of each set of slits 20. The free end of the strap 14 is then inserted through the first slit 20 of the set of slits and disposed around the shaft 24 and then reinserted through the second slit 20 of the set of slits and then secured to the anchored end of the strap 14. The invention could also be practiced by having both ends of the strap free wherein neither of the ends would be anchored to the support member. In this alternative practice of the invention the strap would be inserted through a slit in the support member and looped around the shaft with the two free ends then engaged to each other in any suitable manner, such as by the hook/loop formations. In a further variation one end of the strap could be anchored to the support member 12 and the strap could have fastening structure such as hook/loop formations along a sufficient portion of its length so that the free end of the strap could be engaged at any appropriate location of the strap and not necessarily at a location at the support member. Similarly, neither end of the strap need be anchored to the support member and the securement of the ends of the strap to each other could take place at any location along the circumference of the looped strap and not necessarily at a location at the support member.

As is apparent the key features of the invention include providing a support member which is mounted directly to the penis to, in effect, make the penis erect so that a catheter condom could be reliably placed over the penis for collecting urine. Variations of the structural components are possible within the broad practice of this invention. The drawings illustrate and the corresponding description disclose the preferred practice.

What is claimed is:

1. An assembly for collecting urine from a male comprising a generally rigid shape retaining support member for being disposed against the penis of a male, attaching structure for mounting said support member to the shaft of the penis, and a condom catheter for placement over said support member and the penis for collecting urine from the penis.

2. The assembly of claim 1 wherein said attaching structure comprises at least one strap.

3. The assembly of claim 2 wherein said support member is elongated with a tip end for being disposed generally near the glans penis and a base end for being disposed at the base of the shaft of the penis.

4. The assembly of claim 3 wherein said at least one strap comprises a first strap having one end anchored to said tip end of said support member and a second strap having one end anchored to said base end of said support member.

5. The assembly of claim 4 wherein said tip end of said support member includes a set of at least one slit, said base end of said support member including a set of at least one slit, said first strap having one end anchored to said tip end and having an opposite free end, said first strap being inserted through said set of at least one slit at said tip end, said second strap having one end anchored to said base end and having an opposite free end, and said second strap being inserted through said set of at least one slit at said base end.

6. The assembly of claim 5 wherein each of said sets of at least one slit comprises a first slit and a second slit, each of said straps having an anchored end anchored to the outer surface of said support member at a location between its first slit and second slit, said free end of each of said straps being inserted through said first slit and then back through said second slit and then being secured at said outer surface of said support member.

7. The assembly of claim 6 wherein each of said straps is a soft flexible strap having hook/loop formations at its anchored end and at its free end whereby said free end can be secured to said anchored end.

8. The assembly of claim 7 wherein said support member is concave.

9. The assembly of claim 8 wherein said support member has non-sharp edges.

10. A method of collecting urine from a male patient comprising disposing a support member against the shaft of the penis of the male patient with the support member extending from the base of the shaft to generally near the glans penis, mounting the support member to the shaft of the penis through the use of attaching structure disposed at the support member and extending around the shaft, and placing a condom catheter down and around the support member and the penis for collecting urine from the penis.

11. The method of claim 10 wherein the support member is mounted to the shaft through the use of a first strap anchored at one end to the support member and extending through a first slit at the tip end of the support member and then around the shaft and then through a second slit and then being secured to its anchored end, and further attaching a second strap to the base end of the support member by anchoring one end of the second strap to the support member and then inserting the second strap through a first slit at the base end of the support member and around the shaft and back through a second slit at the base end of the support member and then securing the free end of the strap to the anchored end of the strap.

12. The method of claim 11 wherein the free end of each strap is secured to the anchored end of each strap through the use of engagement of hook/loop formations.
